(19) United States
(12) Patent Application Publication
(10) Pub. No.: US 2010/0143289 A1
(43) Pub. Date: Jun. 10, 2010

(54) UMBILICAL CORD STEM CELL SECRETED PRODUCT DERIVED TOPICAL COMPOSITIONS AND METHODS OF USE THEREOF

(76) Inventors: Michael Cohen, West Orange, NJ (US); Jacob Cohen, West Orange, NJ (US)

Correspondence Address: HOWREY LLP-HN, C/O IP DOCKETING DEPARTMENT, 2941 FAIRVIEW PARK DRIVE, SUITE 200, FALLS CHURCH, VA, 22042-7195, US

(21) Appl. No.: 12/448,458

(22) PCT Filed: Dec. 19, 2007
(86) PCT No.: PCT/US07/25838

§ 371 Date: Feb. 2, 2010

Related U.S. Application Data
(60) Provisional application No. 60/875,558, filed on Dec. 19, 2006.

(51) Int. Cl. A61K 38/19; A61K 35/28; A61K 38/48; A61K 31/19; A61K 38/46; A61K 38/18; A61K 33/24; A61K 33/14; A61K 35/16; A61K 31/195; A61K 31/4406; A61K 31/375; A61K 31/4188; A61K 31/4415; A61K 31/525; A61K 31/51; A61K 31/385; A61K 31/7076; A61K 38/22; A61P 17/02

(52) U.S. Cl. 424/85.1; 424/577; 424/94.64; 514/573; 424/94.67; 514/12; 424/617; 424/665; 424/663; 424/531; 514/561; 514/567; 514/356; 514/474; 514/387; 514/348; 514/251; 514/276; 514/440; 514/557; 514/46

(57) ABSTRACT

Compositions including topical formulations comprising secreted products obtained from the culture medium of human umbilical cord stem cells and particular combinations of components therefrom are provided for treatment of various dermatological conditions, such as adverse consequences of aging, wrinkling, altered pigmentation, altered viscoelasticity, and altered thickness, among others. Methods for using the compositions and topical formulations for treating adverse or undesirable dermatological conditions are also provided, as well as preventing the appearance of undesirable dermatological conditions.

### BACKGROUND

The combination of age, environmental factors and genetic components, among others, frequently contribute to changes in the appearance, viscoelastic properties and topography of the skin and particularly of the face, changes which in many cultures are considered undesirable if not pathologic. While such changes are usually superficial and have no physical impact on the function of the body's major organ, manifestations thereof on the face, neck and to a lesser extent the hands are enduring targets for the application of myriad agents in an age-old attempt to effect a dramatic reversal of or at least arresting progression of the ravages of time thereon. Such attempts and agents are frequently superficial, short term and ineffective.

Human umbilical cord stem cells are cells with a CD45^{neg}, HLA class II^{neg} phenotype. They are stem cells collected from the umbilical cord at birth. Normally, the placenta and its contents are discarded after delivery. It has been found that stem cells can be collected from the umbilical cord before the placenta is discarded. Its current use is to repair the bone marrow after treatments for cancer, as it is thought that umbilical cord stem cells are mainly hematological precursor cells. Until now, stem cells drawn from umbilical cord blood have been reserved mostly for treating children. Because an umbilical cord contains only one-tenth as many stem cells as a marrow donation, it was believed there was too little tissue to reconstitute the immune defenses of an adult. However, new research shows that because the umbilical cord cells proliferate so rapidly, they can indeed be used to treat adults and may even replace bone marrow and other sources of stem cells. Moreover, cord blood transplantation “holds the promise of making it so everyone has a donor.” Cord blood stem cells are collected by hospitals before placentas are discarded and so do not involve the controversy over use of stem cells from fetuses.

### SUMMARY

In one embodiment, a topical composition is provided for the treatment or prevention of various adverse skin conditions, the composition comprising secreted products from umbilical cord stem cells, and a dermatologically suitable topical carrier therefor. In one embodiment, the umbilical cord blood stem cells are adherent, CD45^{neg}, HLA class II^{neg} stem cells. In another embodiment, the adherent, CD45^{neg}, HLA class II^{neg} stem cells are CD34^{pos}. In another embodiment, the adherent, CD45^{neg}, HLA class II^{neg} stem cells are CD34^{neg}, CD106^{neg}, CD44^{pos} and CD90^{pos}. In one embodiment, adverse skin conditions comprise consequences of aging, wrinkling, altered pigmentation, altered viscoelasticity, altered thickness, or any combination of any of the foregoing, by way of non-limiting example.

In another embodiment, a method for treating or preventing adverse skin conditions is provided comprising applying to skin a topical composition comprising secreted products from umbilical cord stem cells, and a dermatologically suitable topical carrier therefor. In one embodiment, the umbilical cord blood stem cells are adherent, CD45^{neg}, HLA class II^{neg} stem cells. In another embodiment, the adherent, CD45^{neg}, HLA class II^{neg} stem cells are CD34^{pos}. In another embodiment, the adherent, CD45^{neg}, HLA class II^{neg} stem cells are CD34^{neg}, CD106^{neg}, CD44^{pos} and CD90^{pos}. In another embodiment, adverse skin conditions comprise consequences of aging, wrinkling, altered pigmentation, altered viscoelasticity, altered thickness, or any combination or any of the foregoing, by way of non-limiting example.

In another embodiment, a topical composition for enhancing properties of the skin or treating adverse skin conditions comprises the following components or homologues or analogues thereof: elastase 2A; prostaglandin I2; prostaglandin E2; adam metallopeptidase with thrombospondin type 1 motif 5; bone morphogenetic protein 1; bone morphogenetic protein 6; chemokine (C—C motif) ligand 2; chemokine (C—C motif) ligand 20; chemokine (C—X—C motif) ligand 1; chemokine (C—X—C motif) ligand 2; chemokine (C—X—C motif) ligand 3; chemokine (C—X—C motif) ligand 5; chemokine (C—X—C motif) ligand 6; chemokine (C—X—C motif) ligand 9; colony stimulating factor 2; colony stimulating factor 3; gremlin 1, cysteine knot superfamily, homolog (Xenopus laevis); gremlin 2, cysteine knot superfamily, homolog (Xenopus laevis); heparin-binding EGF-like growth factor; natriuretic peptide precursor B; pleiotrophin; pre-B-cell colony enhancing factor 1; tumor necrosis factor (ligand) superfamily, member 4; and tumor necrosis factor receptor superfamily, member 11b.

The aforementioned topical composition can be used, in one embodiment, for inducing tumor-like growth of skin cells (without inducing tumorigenesis) or, in another embodiment, increasing the number of keratinocytes in the skin. In another embodiment, the aforementioned compositions can be used to treat aging of skin, wrinkling, altered pigmentation, altered viscoelasticity, altered thickness, or any combination of any of the foregoing. In other embodiments, methods are provided for treating skin with the compositions embodied herein, which, among other activities, induce tumor-like growth of skin cells (without inducing tumorigenesis) or increase the number of keratinocytes in the skin. In another embodiment, methods are provided for treating skin with the compositions embodied herein for addressing adverse skin conditions such as aging, wrinkling, altered pigmentation, altered viscoelasticity, altered thickness, or any combination of any of the foregoing, by way of non-limiting example.

In another embodiment, a topical composition is provided for enhancing properties of the skin or treating adverse skin conditions comprising the following components or homologues or analogues thereof: elastase 2A; prostaglandin I2; prostaglandin E2; amphiregulin; fibroblast growth factor 2; fibroblast growth factor 7; G protein-coupled receptor, family C, group 5, member B; and GABA(a) receptor-associated protein like 1.

The aforementioned topical compositions can be used, in one embodiment, for increasing type V collagen production in the skin, increasing vascularization of the skin; or increasing glandular secretions in the epidermal layer of the skin. In another embodiment, the aforementioned compositions can be used to treat aging, wrinkling, altered pigmentation, altered viscoelasticity, altered thickness, or any combination of any of the foregoing.

The subject matter regarded as the invention is particularly pointed out and distinctly claimed in the concluding portion of the specification. The invention, however, both as to organization and method of operation, together with objects, features, and advantages thereof, may best be understood by reference to the following detailed description.

### DETAILED DESCRIPTION OF THE EMBODIMENTS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

This application provides in one embodiment secreted products from cultures of umbilical cord stem cells that offer benefit in addressing numerous adverse changes that occur in skin during the normal aging process, including but not limited to effects of the environment, such as sunlight, exposure to environmental pollutants, tobacco smoke, and the like, as well as changes having a genetic predisposition. Such adverse changes include but are not limited to adverse pigmentation, wrinkling, loss of elasticity, thinning or skin, among many others. Often such changes do not occur singly but in various combinations, which, in an embodiment, are addressed by the methods and compositions described herein. While such adverse changes are generally superficial with regard to human health, they extract an enormous toll in an individual's self-image and perception by others. Moreover, compositions comprising secreted products are useful for prevention of the appearance of such adverse dermatological conditions. Such secreted products, also called conditioned medium, from umbilical cord stem cells, when formulated into a topically applicable composition, addresses the aforementioned adverse changes or staves off their appearance. Such formulations may be prescription drugs or available over-the-counter or in cosmetic products.

Thus, in one embodiment, a topically-applicable composition is provided for the treatment or prevention of the aforementioned adverse or undesirable changes in skin, the composition comprising at least secreted products from umbilical cord stem cells, and a dermatologically suitable carrier therefor.

In the practice of the embodiments herein, human umbilical cord stem cells are used as a source of the secreted products for the topical compositions herein. They can be grown in accordance with standard protocols, such as, by way of non-limited example, in USSC media (low glucose DMEM with Glutamax, Invitrogen 10567-014), 10⁻⁷ M dexamethasone (Sigma), 100 U/ml penicillin and 0.1 mg/ml streptomycin. In a further example, medium was changed after 48 hrs then every 2-3 days following. On day 14, proliferating cells were passaged 1:3 into new flasks by using 0.25% trypsin/EDTA and neutralized by trypsin neutralization solution. Every 5 to 7 days cells were similarly passaged. The phenotype of passage 5 UCSC was CD31 (2%), CD34 (0%), CD44 (97%), CD50 (0%), CD71 (47%), CD90, (96%), CD106 (0%). The conditioned medium of such cell cultures provides the composition herein for use in various dermatological indications.

In another example, conditioned media was prepared by plating umbilical cord stem cells at 1 million cells per 10 cm plate into 12 mls of HuES media. After 24 hrs media was harvested and sterile filtered.

In other embodiment, umbilical cord blood stem cells used to provide secreted products for the compositions and methods embodied herein are or comprise CD34^{pos} cells.

Secreted products from umbilical cord stem cells is also referred to as “conditioned medium,” a term that refers to a growth medium that is further supplemented by factors derived from media obtained from cultures of cells, in this case, umbilical cord stem cells. The term “effective amount” as used herein is the amount of such described factor as to permit a beneficial effect on skin when formulated and applied as described herein.

In the preparation of a topical composition for the uses herein, the growth medium from umbilical cord stem cells can be used directly, or it may be concentrated, purified, lyophilized, or fractionated, by way of example. In one embodiment, lyophilized condition medium from umbilical cord stem cells is provided in a dermatological composition at a concentration of about 0.001% to about 10%. In another embodiment, conditioned medium from umbilical cord stem cells is formulated directly into a topical formulation, where the conditioned medium comprises from about 1% to about 90% of the composition. As will be noted in other embodiments, fractionation of conditioned medium can be performed and components therein provided in a topical composition. In other embodiment, the secreted products are encapsulated in liposomes which are in turn provided in a formulation for topical application. In such cases the concentration of secreted products within the liposome can be higher and the overall concentration in the topical formulation higher or lower. These are merely illustrative of the formulations and are not meant to be limiting.

In further embodiments, certain combinations of components identified to be present within secreted products from cultures of umbilical cord blood stem cells were found to provide beneficial properties when applied to skin, including treating such adverse skin conditions as consequences of aging, wrinkling, altered pigmentation, altered viscoelasticity, altered thickness, or any combination of any of the foregoing. In other embodiments, benefits were found in that such compositions induced tumor-like growth of skin cells (without inducing tumorigenesis) or increase the number of keratinocytes in the skin. Thus, proliferation of skin cells occurred at a rapid rate but without inducing or promoting a permanent dysproliferative change.

Such compositions comprise, in one embodiment, elastase 2A; prostaglandin I2; prostaglandin E2; adam metallopeptidase with thrombospondin type 1 motif 5; bone morphogenetic protein 1; bone morphogenetic protein 6; chemokine (C—C motif) ligand 2; chemokine (C—C motif) ligand 20; chemokine (C—X—C motif) ligand 1; chemokine (C—X—C motif) ligand 2; chemokine (C—X—C motif) ligand 3; chemokine (C—X—C motif) ligand 5; chemokine (C—X—C motif) ligand 6; chemokine (C—X—C motif) ligand 9; colony stimulating factor 2; colony stimulating factor 3; gremlin 1, cysteine knot superfamily, homolog (Xenopus laevis); gremlin 2, cysteine knot superfamily, homolog (Xenopus laevis); heparin-binding EGF-like growth factor; natriuretic peptide precursor B; pleiotrophin; pre-B-cell colony enhancing factor 1; tumor necrosis factor (ligand) superfamily, member 4; and tumor necrosis factor receptor superfamily, member 11b.

Topical compositions for treatment or prevention of the various conditions described herein may comprise the individual components at a concentration from about 0.000001 to 1.0%. In other embodiments, the components are present in the same relative proportions as they are present in conditioned medium. In a further embodiment the components are present at the same relative proportions as they are present in conditioned medium, but at an overall concentration higher than or lower than present in conditioned medium. As noted herein below, in certain formulations such as a liposome formulation, the aforementioned components may be present at higher amounts within the interior of the liposomes but at a lower concentration in the formulation based on the overall liposome content of the topical formulation.

The aforementioned components of the compositions embodied herein are described in further detail below. The descriptions that are provided are meant to be merely exemplary of the components and biological activities, are not intending to be limiting. A non-limiting list of synonyms and a brief description of the individual components is provided yet Applicant is not bound to the descriptions thereof. Reference is provided to the Uniprot (Universal Protein Resource) data base (www.pir.uniprot.org), a source of sequence and other information on these components. Exemplary, non-limiting information is provided. Homologues of the components include, in the instance where components are proteins, of homologues of various mammalian species, including but not limited to human, chimpanzee, pig, rat, and mouse, as well as muteins and other functional variants, analogs, and modifications of the proteins that provide the same or similar biological activity. Homologues of the organic compounds herein include analogs, variants, adducts, modifications and the like, with the same or similar biological activity as the compound described. Analogs may have enhanced activity.

Elastase 2A (synonyms: ELA1, Elastase-2A precursor, PE-1) is a pancreatic serine protease that hydrolyzes elastin, a fibrous, insoluble protein of connective tissue. Non-limiting examples of elastase 2A include UniProt entries P08217 Q6ISN8, and Q6ISU5, and homologues thereof.

Prostaglandin I2 (synonyms: PGI2, prostacyclin) or 5-[7-hydroxy-8-(3-hydroxyoct-1-enyl)-4-oxabicyclo[3.3.0]oct-3-ylidene] pentanoic acid, is a member of the prostanoids and is known to prevent platelet formation and clumping involved in blood clotting. It is also an effective vasodilator. Non-limiting analogs include iloprost and cisaprost.

Prostaglandin E2 (synonym: PGE2), or (Z)-7-((1R,2R,3R)-3-hydroxy-2-((S,E)-3-hydroxyoct-1-enyl)-5-oxocyclopentyl)hept-5-enoic acid, is a prostanoid that is released by blood vessel walls in response to infection or inflammation that acts on the brain to induce fever.

Adam metallopeptidase with thrombospondin type 1 motif, 5 (synonyms: ADAMTS11, ADAM-TS 11, ADAM-TS5, ADAM-TS 5, ADAMTS-5 precursor, A disintegrin and metalloproteinase with thrombospondin motifs 5, ADMP2, ADMP-2, aggrecanase-2, FLJ36738) is a member of the ADAMTS (a disintegrin and metalloproteinase with thrombospondin motifs) protein family. The enzyme encoded by this gene contains two C-terminal TS motifs and functions as aggrecanase to cleave aggrecan, a major proteoglycan of cartilage. Non-limiting examples include UniProt entries Q9UNA0, Q52LV4, Q9UKP2, and homologues thereof.

Bone morphogenetic protein 1 (synonyms: BMP-1, bone morphogenetic protein 1 precursor, FLJ44432, mammalian tolloid protein, mTld, PCOLC, PCP, procollagen C-proteinase, TLD) is a protein that belongs to the peptidase M12A family of proteins. It induces bone and cartilage development. It is a metalloprotease that cleaves the C-terminus of procollagen I, II and III. It has an astacin-like protease domain. It has been shown to cleave laminin 5 and is localized in the basal epithelial layer of bovine skin. Non-limiting examples include UniProt entries P13497, Q59F71, Q3MIM8, and homologues thereof.

Bone morphogenetic protein 6 (synonyms: BMP-6, bone morphogenetic protein 6 precursor, VGR, VGR1) is a polypetide that is a member of the TGFβ superfamily of proteins. Bone morphogenetic proteins are known for their ability to induce the growth of bone and cartilage. BMP6 is able to induce all osteogenic markers in mesenchymal stem cells. Non-limiting examples include UniProt entries P22004, Q5TCP3, Q4VBA3, and homologues thereof.

Chemokine (C—C motif) ligand 2 (synonyms: GDCF-2, GDCF-2 HC11, HC11, HSMCR30, MCAF, MCP1, MCP-1, MGC9434, monocyte chemoattractant protein 1, monocyte chemotactic and activating factor, monocyte chemotactic protein 1, monocyte secretory protein JE, SCYA2, small inducible cytokine A2 precursor, SMC-CF, monocyte chemotactic protein 1, homologous to mouse Sig-je , small inducible cytokine A2, monocyte chemotactic protein 1, homologous to mouse Sig-je) displays chemotactic activity for monocytes and basophils but not for neutrophils or eosinophils. It has been implicated in the pathogenesis of diseases characterized by monocytic infiltrates, like psoriasis, rheumatoid arthritis and atherosclerosis. It binds to chemokine receptors CCR2 and CCR4. Non-limiting examples include UniProt entries P13500, Q6UZ82, Q9UDF3, and homologues thereof.

Chemokine (C—C motif) ligand 20 (synonyms: Beta chemokine exodus-1, CC chemokine LARC, CKb4, exodus-1, LARC, Liver and activation-regulated chemokine, macrophage inflammatory protein 3 alpha, MIP3A, MIP-3a, MIP-3-alpha, SCYA20, small inducible cytokine A20 precursor, ST38) is a small cytokine belonging to the CC chemokine family. It is strongly chemotactic for lymphocytes and weakly attracts neutrophils. It is implicated in the formation and function of mucosal lymphoid tissues via chemoattraction of lymphocytes and dendritic cells towards the epithelial cells surrounding these tissues. CCL20 elicits its effects on its target cells by binding and activating the chemokine receptor CCR6. Non-limiting examples include UniProt entries P78556, Q53S51, Q99664, and homologues thereof.

Chemokine (C—X—C motif) ligand 1 (synonyms: GRO, GRO1, GROa, GROA, GRO-alpha(1-73), growth-regulated protein alpha precursor, melanoma growth stimulatory activity, MGSA, MGSA-a, MGSA alpha, NAP-3, neutrophil-activating protein 3, SCYB1) is a small cytokine belonging to the CXC chemokine family that was previously called GRO1 oncogene, Neutrophil-activating protein 3 (NAP-3) and melanoma growth stimulating activity, alpha (MSGA-α). It is secreted by human melanoma cells, has mitogenic properties and is implicated in melanoma pathogenesis. CXCL1 is expressed by macrophages, neutrophils and epithelial cells, and has neutrophil chemoattractant activity. CXCL1 plays a role in spinal cord development by inhibiting the migration of oligodendrocyte precursors and is involved in the processes of angiogenesis, inflammation, wound healing, and tumorigenesis. This chemokine elicits its effects by signaling through the chemokine receptor CXCR2. Non-limited examples include UniProt entries P09341, Q6LD34, and homologues thereof.

Chemokine (C—X—C motif) ligand 2 (synonyms: CINC-2a, GRO2, GROb, GROB, Gro-beta, Growth-regulated protein beta, Macrophage inflammatory protein 2-alpha precursor, MGSA-b, MGSA beta, MIP2, MIP2A, MIP-2a, MIP2-alpha, SCYB2) is a small cytokine belonging to the CXC chemokine family that is also called macrophage inflammatory protein 2-alpha (MIP2-alpha), Growth-regulated protein beta (Gro-beta) and Gro oncogene-2 (Gro-2). CXCL2 is 90% identical in amino acid sequence as a related chemokine, CXCL1. This chemokine is secreted by monocytes and macrophages and is chemotactic for polymorphonuclear leukocytes and hematopoietic stem cells. The gene for CXCL2 is located on human chromosome 4 in a cluster of other CXC chemokines. CXCL2 mobilizes cells by interacting with a cell surface chemokine receptor called CXCR2. Non-limiting examples include UniProt entries P19875, Q6LD33, Q6FGD6, and homologues thereof.

Chemokine (C—X—C motif) ligand 3 (synonyms: CINC-2b, GRO3, GROg, GROG, GRO-gamma, GRO-gamma(1-73), growth-regulated protein gamma, macrophage inflammatory protein 2-beta precursor, MGSA gamma, MIP2B, MIP-2b, MIP2-beta, SCYB3) is a small cytokine belonging to the CXC chemokine family that is also known as GRO3 oncogene (GRO3), GRO protein gamma (GROg) and macrophage inflammatory protein-2-beta (MIP2b). CXCL3 controls migration and adhesion of monocytes and mediates it effects on its target cell by interacting with a cell surface chemokine receptor called CXCR2. Non-limiting examples include UniProt entries P19876, Q6LD32, Q4W5H9, and homologues thereof.

Chemokine (C—X—C motif) ligand 5 (synonyms: ENA78, ENA-78, ENA-78(1-78), epithelial-derived neutrophil-activating protein 78, neutrophil-activating peptide ENA-78, SCYB5, small inducible cytokine B5 precursor) is a small cytokine belonging to the CXC chemokine family that is also known as epithelial-derived neutrophil-activating peptide 78 (ENA-78). It is produced following stimulation of cells with the inflammatory cytokines interleukin-1 or tumor necrosis factor-alpha. Expression of CXCL5 has also been observed in eosinophils, and can be inhibited with the type II interferon IFN-γ. This chemokine stimulates the chemotaxis of neutrophils possessing angiogenic properties. It elicits these effects by interacting with the cell surface chemokine receptor CXCR2. CXCL5 has been implicated in connective tissue remodelling. Non-limiting examples include UniProt entries P42830, Q6I9S7, Q96QE1, and homologues thereof.

Chemokine (C—X—C motif) ligand 6 (synonyms: chemokine alpha 3, CKA-3, GCP2, GCP-2, granulocyte chemotactic protein 2, SCYB6, small inducible cytokine B6 precursor) is a small cytokine belonging to the CXC chemokine family that is also known as granulocyte chemotactic protein 2 (GCP-2). As its former name suggests, CXCL6 is a chemoattractant for neutrophilic granulocytes. It elicits its chemotactic effects by interacting with the chemokine receptors CXCR1 and CXCR2. Non-limiting examples include UniProt entries P80162, Q4W5D4, O00172, and homologues thereof.

Chemokine (C—X—C motif) ligand 9 (synonyms: CMK, crg-10, Gamma interferon-induced monokine, Humig, MIG, SCYB9, Small inducible cytokine B9 precursor) is a small cytokine belonging to the CXC chemokine family that is also known as Monokine induced by gamma interferon (MIG). CXCL9 is a T-cell chemoattractant, which is induced by IFN-γ. CXCL9 elicits its chemotactic function by interacting with the chemokine receptor CXCR3. Non-limiting examples include UniProt entries Q07325, Q503B4, and homologues thereof.

Colony stimulating factor 2 (granulocyte-macrophage) (synonyms: colony-stimulating factor, CSF, GMCSF, GM-CSF, granulocyte-macrophage colony-stimulating factor precursor, MGC131935, molgramostin, sargramostim) is a cytokine that functions as a white blood cell growth factor. GM-CSF stimulates stem cells to produce granulocytes (neutrophils, eosinophils, and basophils) and monocytes. Monocytes exit the circulation and migrate into tissue, whereupon they mature into macrophages. It is thus part of the immune/inflammatory cascade, by which activation of a small number of macrophages can rapidly lead to an increase in their numbers, a process crucial for fighting infection. The active form of the protein is found extracellularly as a homodimer. Non-limiting examples include UniProt entries P04141, Q647J8, Q2VPI8, and homologues thereof.

Colony stimulating factor 3 (granulocyte) (synonyms: filgrastim, GCSF, G-CSF, granulocyte colony-stimulating factor precursor, lenograstim, MGC45931, pluripoietin) is a growth factor or cytokine produced by a number of different tissues to stimulate the bone marrow to produce granulocytes and stem cells. G-CSF then stimulates the bone marrow to pulse them out of the marrow into the blood. It also stimulates the survival, proliferation, differentiation, and function of neutrophil precursors and mature neutrophils. Non-limiting examples include UniProt entries P09919, Q8N4W3, Q6FH65, and homologues thereof.

Gremlin 1, cysteine knot superfamily, homolog (Xenopus laevis) (synonyms: CKTSF1B1, Cysteine knot superfamily 1, BMP antagonist 1, DAND2, down-regulated in Mos-transformed cells protein, DRM, gremlin, GREMLIN, gremlin-1 precursor, IHG-2, increased in high glucose protein 2, MGC126660, PIG2, proliferation-inducing gene 2 protein) is a member of the BMP (bone morphogenic protein) antagonist family. Like BMPs, BMP antagonists contain cystine knots and typically form homo- and heterodimers. The antagonistic effect of the secreted glycosylated protein is likely due to its direct binding to BMP proteins. Non-limiting examples include UniProt entries O60565, Q52LV3, Q8N936, and homologues thereof.

Gremlin 2, cysteine knot superfamily, homolog (Xenopus laevis) (synonyms: CKTSF1B2, Cysteine knot superfamily 1, BMP antagonist 2, DAND3, FLJ21195, gremlin-2 precursor, Prdc, PRDC, protein related to DAN and cerberus) is a cytokine that inhibits the activity of BMP2 and BMP4 in a dose-dependent manner. It antagonizes BMP4-induced suppression of progesterone production in granulosa cells. Non-limiting examples include UniProt entries Q9H772 and Q86UD9, and homologues thereof.

Heparin-binding EGF-like growth factor (synonyms: Diphtheria toxin receptor, DTR, DT-R, DTS, HB-EGF, HEGFL, heparin-binding EGF-like growth factor precursor) is a member of the EGF family of proteins. It has been shown to play a role in wound healing, cardiac hypertrophy and heart development and function. Non-limiting examples include UniProt entries Q99075, Q9UMJ6, Q53H93, and homologues thereof.

Natriuretic peptide precursor B (synonym: brain natriuretic peptide, B-type natriuretic peptide, GC-B) is a 32 amino acid polypeptide secreted by the ventricles of the heart in response to excessive stretching of myocytes (heart muscles cells) in the ventricles. At the time of release, a co-secreted 76 amino acid N-terminal fragment (NT-proBNP) is also released with BNP. BNP binds to and activates NPRA in a similar fashion to atrial natriuretic peptide (ANP) but with 10-fold lower affinity. The biological half-life of BNP, however, is twice as long as that of ANP. Both ANP and BNP have limited ability to bind and activate NPRB. Physiologic actions of BNP and ANP include decrease in systemic vascular resistance and central venous pressure as well as an increase in natriuresis. Thus, the resulting effect of these peptides is a decrease in cardiac output and a decrease in blood volume. Non-limiting examples include UniProt entries P16860, Q6FGY0, Q9P2Q7, and homologues thereof.

Pleiotrophin (synonyms: HARP, HBBM, HB-GAM, HBGF8, HBGF-8, HBNF, HBNF1, HBNF-1, heparin-binding brain mitogen, heparin-binding growth-associated molecule, heparin-binding growth factor 8, heparin-binding neurite outgrowth-promoting factor 1, NEGF1, OSF-1, osteoblast-specific factor 1, pleiotrophin precursor) is an 18-kDa growth factor that has a high affinity for heparin. Pleiotrophin was initially recognized as a neurite outgrowth-promoting factor present in rat brain around birth and as a mitogen toward fibroblasts isolated from bovine uterus tissue. Non-limiting examples include UniProt entries P21246, Q5U0B0, Q6ICQ5, and homologues thereof.

Pre-B-cell colony enhancing factor 1 (synonyms: MGC117256, NAmPRTase, Nampt, NAMPT, nicotinamide phosphoribosyltransferase, PBEF, pre-B-cell colony-enhancing factor 1, pre-B cell-enhancing factor, visfatin) is a nicotinamide phosphoribosyltransferase (Nampt) enzyme that catalyzes first step in the biosynthesis of NAD from nicotinamide. This protein has also been reported to be a cytokine (PBEF) that promotes B cell maturation and inhibits neutrophil apoptosis. It enhances the maturation of B cell precursors in the presence of Interleukin (IL)-7 and stem cell factor. Non-limiting examples include UniProt entries P43490, Q3KQV0, Q8WW95, and homologues thereof.

Tumor necrosis factor (ligand) superfamily, member 4 (tax-transcriptionally activated glycoprotein 1, 34 kDa) (synonyms: CD134L, CD252, CD252 antigen, Glycoprotein Gp34, gp34, GP34, OX40L, OX-40L, OX40 ligand, OX4OL, TAX transcriptionally-activated glycoprotein 1, tumor necrosis factor ligand superfamily member 4, TXGP1) is a cytokine that belongs to the tumor necrosis factor (TNF) ligand family. This cytokine is a ligand for receptor TNFRSF4/OX4. It is found to be involved in T cell antigen-presenting cell (APC) interactions. In surface Ig- and CD40-stimulated B cells, this cytokine along with CD70 has been shown to provide CD28-independent costimulatory signals to T cells. This protein and its receptor are reported to directly mediate adhesion of activated T cells to vascular endothelial cells. Non-limiting examples include UniProt entries P23510, Q8IV74, Q5JZA5, and homologues thereof.

Tumor necrosis factor receptor superfamily, member 11b (synonyms: MGC29565, OCIF, OPG, osteoclastogenesis inhibitory factor, osteoprotegerin, TR1, tumor necrosis factor receptor superfamily member 11B precursor) is a cytokine and a member of the tumor necrosis factor (TNF) receptor superfamily. It inhibits the differentiation of macrophages into osteoclasts and also regulates the resorption of osteoclasts in vitro and in vivo. Osteoprotegerin is a RANK homolog, and works by binding to RANK ligand on osteoblast/stromal cells, thus blocking the RANK-RANK ligand interaction between osteoblast/stromal cells and osteoclast precursors. This has the effect of inhibiting the differentiation of the osteoclast precursor into a mature osteoclast. Non-limiting examples include UniProt entries O00300, Q53FX6, O60236, and homologues thereof.

In further embodiments, certain combinations of components identified to be present within secreted products from cultures of umbilical cord blood stem cells were found to provide beneficial properties when applied to skin, including treating such adverse skin conditions as consequences of aging, wrinkling, altered pigmentation, altered viscoelasticity, altered thickness, or any combination of any of the foregoing. In other embodiments, the components were found to increase type V collagen production in the skin, increase vascularization of the skin; or increase glandular secretions in the epidermal layer of the skin. Any one or two, or all three, of the aforementioned activities are embraced in other embodiments.

Such compositions comprise, in one embodiment, elastase 2A; prostaglandin I2; prostaglandin E2; amphiregulin; fibroblast growth factor 2; fibroblast growth factor 7; G protein-coupled receptor, family C, group 5, member B; and GABA(a) receptor-associated protein like 1.

Topical compositions for treatment or prevention of the various conditions described herein may comprise the individual components at a concentration from about 0.000001 to 1.0%. In other embodiments, the components are present in the same relative proportions as they are present in conditioned medium. In a further embodiment the components are present at the same relative proportions as they are present in conditioned medium, but at an overall concentration higher than or lower than present in conditioned medium. As noted herein below, in certain formulations such as a liposome formulation, the aforementioned components may be present at higher amounts within the interior of the liposome but at a lower concentration in the formulation based on the overall liposome content of the topical formulation.

The aforementioned components of the compositions embodied herein are described in further detail below. The descriptions that are provided are meant to be merely exemplary of the components and biological activities, are not intending to be limiting. A non-limiting list of synonyms and a brief description of the individual components is provided yet Applicant is not bound to the descriptions thereof. Reference is provided to the Uniprot (Universal Protein Resource) data base (www.pir.uniprot.org), a source of sequence and other information on these components. Exemplary, non-limiting information is provided. Homologues of the components include, in the instance where components are proteins, of homologues of various mammalian species, including but not limited to human, chimpanzee, pig, rat, and mouse, as well as muteins and other functional variants, analogs, and modifications of the proteins that provide the same or similar biological activity. Homologues of the organic compounds herein include analogs, variants, adducts, modifications and the like, with the same or similar biological activity as the compound described. Analogs may have enhanced activity.

Elastase 2A, prostaglandin I2 and prostaglandin E2 are described above.

Amphiregulin (synonyms: amphiregulin precursor, AR, schwannoma-derived growth factor, Sdgf, SDGF) is a member of the EGF family of proteinsm and is a major autocrine growth factor for cultured human keratinocytes and probably plays a role in the aberrant keratinocyte growth of hyperproliferative disorders. Non-limiting examples include UniProt entry P24338 and homologues thereof.

Fibroblast growth factor 2 (synonyms: basic fibroblast growth factor, FGF2, BFGF, FGFB, HBGF-2, HBGH-2, heparin-binding growth factor 2 precursor, prostatropin) is a member of the fibroblast growth factor family. In normal tissue, basic fibroblast growth factor is present in basement membranes and in the subendothelial extracellular matrix of blood vessels. It stays membrane-bound as long as there is no signal peptide. Non-limiting examples include UniProt entries P09038, Q9UC54, and Q7KZ72, and homologues thereof.

Fibroblast growth factor 7 (synonyms: FGF-7, HBGF-7, keratinocyte growth factor precursor, KGF) is a member of the fibroblast growth factor family that stimulates the growth of epithelial cells, but lacks mitogenic activity on fibroblasts or endothelial cells. FGF7 is a single polypeptide chain of about 28 kD that has predominant activity in keratinocytes. Non-limiting examples include UniProt entries P21781, Q6RK68, Q6FGV5, and homologues thereof.

G protein-coupled receptor, family c, group 5, member b (synonyms: GPRC5B, G-protein coupled receptor family C group 5 member B precursor, RAIG2, RAIG-2, retinoic acid-induced gene 2 protein) is characterized by a signature 7-transmembrane domain motif. The specific function of this protein is unknown; however, this protein may mediate the cellular effects of retinoic acid on the G protein signal transduction cascade. The protein encoded by this gene is a member of the type 3 G protein-coupled receptor family. Non-limiting examples include UniProt entries Q9NZH0, O75205, Q8NBZ8, and homologues thereof.

GABA(a) receptor-associated protein like 1 (synonyms: APG8L, ATG8, early estrogen-regulated protein, GABA(A) receptor-associated protein-like 1, gamma-aminobutyric acid receptor-associated protein-like 1, gec1, GEC1, GEC-1, glandular epithelial cell protein 1) was described by Vernier-Magnin et al., 2001, A novel early estrogen-regulated gene gec1 encodes a protein related to GABARAP. Biochem Biophys Res Commun. 284:118-25. Non-limiting examples include UniProt entries Q9H0R8 and Q6FIE6, and homologues thereof.

Thus in the foregoing embodiments particular components identified in secreted products from cultures of umbilical cord stem cells offer benefit in addressing numerous adverse changes that occur in skin during the normal aging process, including but not limited to effects of the environment, such as sunlight, exposure to environmental pollutants, tobacco smoke, and the like, as well as changes having a genetic predisposition. Such adverse changes include but are not limited to adverse pigmentation, wrinkling, loss of elasticity, thinning or skin, among many others. Often such changes do not occur singly but in various combinations, which, in an embodiment, are addressed by the methods and compositions described herein. While such adverse changes are generally superficial with regard to human health, they extract an enormous toll in an individual's self-image and perception by others. Moreover, compositions comprising the particular components of secreted products are useful for prevention of the appearance of such adverse dermatological conditions. Such components, when formulated into a topically applicable compositions, address the aforementioned adverse changes or stave off their appearance. Such formulations may be prescription drugs or available over-the-counter or in cosmetic products.

Thus, in one embodiment, a topically-applicable composition is provided for the treatment or prevention of the aforementioned adverse or undesirable changes in skin, the composition comprising at least particular components from secreted products from umbilical cord stem cells, and a dermatologically suitable carrier therefor.

To prepare a composition comprising any of the compositions described previously, the individual components may be purchased from commercial suppliers that sell proteins and other biochemicals, or expressed from constructs prepared from purchased cDNA or identified from a cDNA library. In other embodiments, the proteins can be isolated from embryonic germ cell derivatives conditioned medium or other conditioned medium from cells that produce the desired component, or purified from other biological sources. Other means of procuring or producing the components are well known to the skilled artisan. Companies that sell biochemicals, proteins, and cDNAs include Sigma Life Sciences, Invitrogen, OriGene Technologies, which are merely a few examples of numerous commercial sources.

In further embodiments, any of the foregoing compositions can optionally also include the following components: MgSO₄ (anhydrous); CaCl₂ (anhydrous); KCl; NaCl; NaHCO₃; NaH₂PO₄.H₂O; L-alanine; L-arginine.HCl; L-asparagine.H₂O; L-aspartic acid; L-cysteine.HCl.H₂O; L-cystine.2HCl; L-glutamic acid; L-glutamine; glycine; L-histidine.HCl.H₂O; L-isoleucine; L-leucine; L-lysine.HCl; L-methionine; L-phenylalanine; L-proline; L-serine; L-threonine; L-tryptophan; L-tyrosine.2Na.2H₂O; L-valine; ascorbic acid; biotin; D-calcium pantothenate; i-inositol; nicotinamide; pyridoxine.HCl; riboflavin; thiamine.HCl; vitamin B12; choline chloride; folic acid; D-glucose; lipoic acid; sodium pyruvate; thymidine; adenosine; cytidine; guanosine; uridine; 2′-deoxyadenosine; 2′-deoxycytidine.HCl; 2′-deoxyguanosine; and fetal calf serum. In other embodiments, the concentrations of the components are as follows: MgSO_{4 (anhydrous;} 97.67 mg/L); CaCl₂ (anhydrous; 200 mg/L); KCl (400 mg/L); NaCl (6800 mg/L); NaHCO₃ (2200 mg/L); NaH₂PO₄.H₂O (140 mg/L); L-alanine (25 mg/L); L-arginine.HCl (105 mg/L); L-asparagine.H₂O (50 mg/L); L-aspartic acid (30 mg/L); L-cysteine.HCl.H₂O (100 mg/L); L-cystine.2HCl (31 mg/L); L-glutamic acid (75 mg/L); L-glutamine (292 mg/L); glycine (50 mg/L); L-histidine.HCl.H₂O (31 mg/L); L-isoleucine (52.4 mg/L); L-leucine (52 mg/L); L-lysine.HCl (73 mg/L); L-methionine (15 mg/L); L-phenylalanine (32 mg/L); L-proline (40 mg/L); L-serine (25 mg/L); L-threonine (48 mg/L); L-tryptophan (10 mgL); L-tyrosine.2Na.2H₂O (52 mg/L); L-valine (46 mg/L); ascorbic acid (50 mg/L); biotin (0.1 mg/L); D-calcium pantothenate (1 mg/L); i-inositol (2 mg/L); nicotinamide (1 mg/L); pyridoxine.HCl (1 mg/L); riboflavin (0.1 mg/L); thiamine.HCl (1 mg/L); vitamin B₁₂ (1.36 mg/L); choline chloride (1 mg/L); folic acid (1 mg/L); D-glucose (1000 mg/L); lipoic acid (0.2 mg/L); sodium pyruvate (110 mg/L); thymidine (10 mg/L); adenosine (10 mg/L); cytidine (10 mg/L); guanosine (10 mg/L); uridine (10 mg/L); 2′-deoxyadenosine (10 mg/L); 2′-deoxycytidine.HCl (11 mg/L); 2′-deoxyguanosine (10 mg/L); and fetal calf serum (2% v/v). In a further embodiment the aforementioned composition can also contain 1% of a 100× solution of glutamine-penicillin-streptomycin (where the 100× solution contains, in one milliliter 10 mM citrate buffer, 10,000 units of penicillin base [as penicillin G, sodium salt], 10 mg of streptomycin base [as streptomycin sulfate], and 29.2 mg of L-glutamine). The foregoing concentrations may be proportionally diluted or concentrated in other embodiments herein.

In certain embodiments, the aforementioned culture medium components are present at a concentration from that used to grow umbilical cord blood stem cells to about 100-fold less. In one embodiment, for example, fetal calf serum is present at about 2%, the same concentration present in culture medium, or as low as 0.02% (v/v). The foregoing concentrations may be proportionally diluted or concentrated in other embodiments herein.

In one embodiment, the aforementioned compositions are provided in a liposome or microencapsulated formulation contained within a topical carrier, for application to the skin. Liposomal formulations are well known in the art, including those for topical formulations. Liposome delivery has been utilized as a pharmaceutical delivery system for many for a variety of applications [see Langer, Science, 1990, 249:1527-1533; Treat et al., in Liposomes in the Therapy of Infectious Disease and Cancer, Lopez-Berenstein and Fidler (eds.), Liss: New York, pp-353-365 (1989); Lopez-Berenstein, ibid., pp. 317-327]. A general discussion of liposomes and liposome technology can be found in a three volume work entitled “Liposome Technology” edited by G. Gregoriadis, 1993, published by CRC Press, Boca Raton, Fla. In one non-limiting embodiment, a liposome composed of phosphatidyl choline, phosphatidyl ethanolamine, oleic acid and cholesteryl hemisuccinate is used. The liposome-encapsulated composition can also be combined with an aesthetically or pharmaceutically-acceptable base for topical application, as described below.

The secreted products or compositions described above can be provided in a composition comprising at least one dermatologically suitable carrier. Suitable carriers include such components as emollients, emulsifiers, diluents, preservatives, solubilizers and/or carriers. In one typical embodiment, a hydrogel carrier is used. In one embodiment the formulation is applied as a liquid or gel to the skin and rubbed in by the user, or spread on the skin and occluded by a bandage or other device to maintain contact for a period of time. In another embodiments, a semisolid hydrogel formulation (akin to a wound dressing) comprising secreted products is placed on the affected areas of the skin, and allowed to remain in place as secreted products interact with the skin. It may be covered with an occlusive bandage or other device to maintain moisture. After a sufficient period of time, the hydrogel material is removed and discarded.

In one embodiment, secreted products from, for example, frozen human umbilical cord blood mononuclear cells from Cambrex, are used.

The compositions herein may contain a wide range of additional, optional components. The CTFA Cosmetic Ingredient Handbook, Seventh Edition, 1997 and the Eighth Edition, 2000, which is incorporated by reference herein in its entirety, describes a wide variety of cosmetic and pharmaceutical ingredients commonly used in skin care compositions, which are suitable for use in the compositions herein. Examples of these functional classes disclosed in this reference include: absorbents, abrasives, anticaking agents, antifoaming agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, preservatives, propellants, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), sunscreen agents, ultraviolet light absorbers, waterproofing agents, and viscosity increasing agents (aqueous and nonaqueous).

Water is employed in amounts effective to form an emulsion. It is generally desirable to use water which has been purified by processes such as deionization or reverse osmosis, to improve the batch-to-batch formulation inconsistencies which can be caused by dissolved solids in the water supply. The amount of water in the emulsion or composition can range from about 15 percent to 95 weight percent, usually from about 45 to 75 percent, and typically from about 60 percent to about 75 percent.

An emollient is an oleaginous or oily substance which helps to smooth and soften the skin, and may also reduce its roughness, cracking or irritation. Typical suitable emollients include mineral oil having a viscosity in the range of 50 to 500 centipoise (cps), lanolin oil, coconut oil, cocoa butter, olive oil, almond oil, macadamia nut oil, aloe extracts such as aloe vera lipoquinone, synthetic jojoba oils, natural Sonora jojoba oils, safflower oil, corn oil, liquid lanolin, cottonseed oil and peanut oil. In one embodiment, the emollient is a cocoglyceride, which is a mixture of mono, di and triglycerides of cocoa oil, sold under the trade name of Myritol 331 from Henkel KGaA, or Dicaprylyl Ether available under the trade name Cetiol OE from Henkel KGaA or a C₁₂₋₁₅ alkyl benzoate sold under the trade name Finsolv TN from Finetex. One or more emollients may be present ranging in amounts from about 1 percent to about 10 percent by weight, typically about 5 percent by weight. Another suitable emollient is DC 200 Fluid 350, a silicone fluid, available Dow Corning Corp.

Other suitable emollients include squalane, castor oil, polybutene, sweet almond oil, avocado oil, calophyllum oil, ricin oil, vitamin E acetate, olive oil, silicone oils such as dimethylopolysiloxane and cyclomethicone, linolenic alcohol, oleyl alcohol, the oil of cereal germs such as the oil of wheat germ, isopropyl palmitate, octyl palmitate, isopropyl myristate, hexadecyl stearate, butyl stearate, decyl oleate, acetyl glycerides, the octanoates and benzoates of C₁₂₋₁₅ alcohols, the octanoates and decanoates of alcohols and polyalcohols such as those of glycol and glyceryl, ricinoleates esters such as isopropyl adipate, hexyl laurate and octyl dodecanoate, dicaprylyl maleate, hydrogenated vegetable oil, phenyltrimethicone, jojoba oil and aloe vera extract.

Other suitable emollients which are solids or semi-solids at ambient temperatures may be used. Such solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate. One or more emollients can optionally be included in the formulation.

A humectant is a moistening agent that promotes retention of water due to its hygroscopic properties. Suitable humectants include glycerin, polymeric glycols such as polyethylene glycol and polypropylene glycol, mannitol and sorbitol. Typically, the humectant is sorbitol, 70% USP or polyethylene glycol 400, NF. One or more humectants can optionally be included in the formulation in amounts from about 1 percent to about 10 percent by weight, typically about 5 percent by weight.

A hydrogel composition can also be used, and can include a biocompatible polymer component. The biocompatible polymer component can include one or more natural polymers, synthetic polymers, or combinations thereof. For example, the biocompatible polymer can be a polyalkylene oxide such as polyethylene glycol (PEG) or polypropylene glycol, or a derivative of PEG including but not limited to carbonates of polyethylene glycol. The hydrogel can be non-ionic, cationic or anionic. Many other hydrogel-forming polymers are known to the skilled practitioner, including those employing monomeric saccharides, amino acids, and others, to name only an exemplary few. Furthermore, various physicochemical properties are known for hydrogels, such as liquids, pastes, and membranes that can be applied to skin, for example. Various other non-limiting examples are described in US Patent Application 20050112151, incorporated herein by reference.

A dry-feel modifier is an agent which when added to an emulsion, imparts a “dry feel” to the skin when the emulsion dries. Dry feel modifiers can include talc, kaolin, chalk, zinc oxide, silicone fluids, inorganic salts such as barium sulfate, surface treated silica, precipitated silica, fumed silica such as an Aerosil available from Degussa Inc. of New York, N.Y. U.S.A.

It may be advantageous to incorporate additional thickening agents, such as, for instance, Carbopol Ultrez, or alternatively, Carbopol ETD 2001, available from the B.F. Goodrich Co. The selection of additional thickening agents is well within the skill of one in the art.

A waterproofing or water resistance agent is a hydrophobic material that imparts film forming and waterproofing characteristics to an emulsion. A suitable waterproofing agent is a copolymer of vinyl pyrollidone and eicosene and dodecane monomers such as Ganex V 220 and Ganex V 216 Polymers, respectively, trade names of ISP Inc. of Wayne, N.J. U.S.A. Still other suitable waterproofing agents include polyurethane polymer, such as Performa V 825 available from New Phase Technologies and polyanhydride resin No. 18 available under the trade name PA-18 from Chevron. The waterproofing agent is used in amounts effective to allow the formulation embodied herein to remain effective on the skin after exposure to circulating water for at least 40 minutes for water resistance and at least 80 minutes for waterproofing using the procedures described by the U.S. Food and Drug Administration in “Sunscreen Drug Products for OTC Human Use,” Federal Register, Vol. 43, Aug. 25, 1978, Part 2, pp. 38206-38269.

An antimicrobial preservative is a substance or preparation which destroys, or prevents or inhibits the proliferation of, microorganisms in the composition, and which may also offer protection from oxidation. Preservatives are frequently used to make self-sterilizing, aqueous based products such as emulsions. This is done to prevent the development of microorganisms that may be in the product from growing during manufacturing and distribution of the product and during use by consumers, who may further inadvertently contaminate the products during normal use. Typical preservatives include the lower alkyl esters of para-hydroxybenzoates (parabens), especially methylparaben, propylparaben, isobutylparaben and mixtures thereof, benzyl alcohol, phenyl ethyl alcohol and benzoic acid. One preservative is available under the trade name of Germaben II from Sutton. One or more antimicrobial preservatives can optionally be included in an amount ranging from about 0.001 to about 10 weight percent, typically about 0.05 to about 1 percent.

An antioxidant is a natural or synthetic substance added to the composition herein to protect from or delay its deterioration due to the action of oxygen in the air (oxidation). Anti-oxidants prevent oxidative deterioration which may lead to the generation of rancidity and nonenyzymatic browning reaction products. Typical suitable antioxidants include propyl, octyl and dodecyl esters of gallic acid, butylated hydroxyanisole (BHA, usually purchased as a mixture of ortho and meta isomers), butylated hydroxytoluene (BHT), nordihydroguaiaretic acid, Vitamin A, Vitamin E and Vitamin C. One or more antioxidants can optionally be included in the composition in an amount ranging from about 0.001 to about 5 weight percent, typically about 0.01 to about 0.5 percent.

Chelating agents are substances used to chelate or bind metallic ions, such as with a heterocyclic ring structure so that the ion is held by chemical bonds from each of the participating rings. Suitable chelating agents include ethylene diaminetetraacetic acid (EDTA), EDTA disodium, calcium disodium edetate, EDTA trisodium, EDTA tetrasodium and EDTA dipotassium. One or more chelating agents can optionally be included in the composition embodied herein in amounts ranging from about 0.001 to about 0.2 weight percent typically about 0.01% weight percent.

Fragrances are aromatic substances which can impart an aesthetically pleasing aroma to the composition. Typical fragrances include aromatic materials extracted from botanical sources (i.e., rose petals, gardenia blossoms, jasmine flowers, etc.) which can be used alone or in any combination to create essential oils. Alternatively, alcoholic extracts may be prepared for compounding fragrances. However, due to the relatively high costs of obtaining fragrances from natural substances, the modem trend is to use synthetically prepared fragrances, particularly in high-volume products. The fragrances for use herein may be Fragrance SZ-2108 and Fragrance SZ-1405 available from Sozio, Inc. One or more fragrances can optionally be included in the composition herein in an amount ranging from about 0.001 to about 5 weight percent, typically about 0.01 to about 0.5 percent by weight.

A pH modifier is a compound that will adjust the pH of a formulation to a lower, e.g., more acidic pH value, or to a higher, e.g., more basic pH value. The selection of a suitable pH modifier is well within the ordinary skill of one in the art.

Suitable emulsifiers for one aspect of the embodiments herein are those known in the art for producing oil-in-water and/or water-in-oil type emulsions. An aqueous external phase is preferred by many people for skin contact, since it is not as likely to produce an oily or greasy sensation when it is being applied, as is an emulsion having an oil external phase. The typical oil-in-water emulsifier has a hydrophilic-lipophilic balance (frequently abbreviated as “HLB”) value greater than about 9, as is well known in the art; however, this “rule” is known to have numerous exceptions. The chosen emulsifier, depending upon its chemical nature, will be a component of either the oil or aqueous phase, and assists with both the formation and the maintenance, or stability, of the emulsion. Suitable emulsifiers for another embodiment herein are those known in the art for producing water-in-oil type emulsions. The typical water-in-oil emulsifier has a HLB value of about 4 to about 6, as is well known in the art; however, this “rule” is also known to have numerous exceptions. Selection of suitable water-in-oil emulsifiers is well known in the formulation art.

Most of the widely used emulsifier systems for formulations embodied herein can be used in the formulations herein. In some embodiment, emulsifiers are PEG-8 Distearate available under the trade name of Emerest 2712 from Henkel, PEG-5 Glyceryl Stearate available under the trade name POEM-S-105 from Riken Vitamin Oil, PEG-6 Hydrogenated Castor Oil, available under the trade name Sabowax ELH6 from Sabo, PEG-6 Oleate, available under the trade name STEPAN PEG-300 MO from Stepan, Sorbitan Sesquioleate, available under the trade name Arlacel 83 and Arlacel C from ICI Surfactants, TEA-Stearate, available under the trade name of Cetasal from Gaftefosse S.A. Another typical emulsifier is neutralized cetyl phosphate, available under the trade name Amphisol A from LaRoche. In another embodiment the emulsifier is an Acrylate/C₁₀₋₃₀ alkyl acrylate cross polymer of C₁₀₋₃₀ alkyl acrylates and one or more monomers of acrylic acid, methacrylic acid or one of their simple esters crosslinked within allyl ether of sucrose or an allyl ether of pentaerythritol, available under the trade names of Pemulen TR from B.F. Goodrich. The amount of emulsifier used herein may present in an amount of about 0.1 to about 10% by weight, typically about 0.5 percent to about 5 percent by weight, most typically about 2 percent to about 4 percent by weight. The choice of an emulsifier is well within ordinary skill in the art and is not a critical aspect herein. Additional emulsifiers that may be employed include sorbitan triisostearate available under the trade name Crill 6 from Croda Oleochemicals, and polyglyceryl-3 distearate available under the trade name Cremophor GS 32 from BASF.

As is known in the art, the individual emulsion droplets typically have a small and uniform size because these properties result in a more stable emulsion. Conversely, a broad particle size distribution indicates that the interfacial tension between the droplets has not been substantially reduced, and thus the droplets tend to coalesce and form agglomerations that result in an unstable emulsion.

Further to the discussion above, hydrogels may comprise poly(N-vinyl lactam), including homopolymers, copolymers and terpolymers of N-vinyl lactams such as N-vinylpyrrolidone, N-vinylbutyrolactam, N-vinylcaprolactam, and the like, as well as the foregoing prepared with minor amounts, for example, up to about 50 weight percent, of one of a mixture of other vinyl monomers copolymerizable with the N-vinyl lactams. Copolymers or terpolymers of poly (N-vinyl-lactam) may comprise N-vinyl-lactam monomers such as vinylpyrrolidone copolymerized with monomers containing a vinyl functional group such as acrylates, hydroxyalkylacrylates, methacrylates, acrylic acid or methacrylic acid, and acrylamides. Of the poly(N-vinyl lactam) homopolymers, the polyvinylpyrrolidone (PVP) homopolymers are preferred. Of the poly(N-vinyl lactam) copolymers, the vinyl pyrrolidone and acrylamide copolymers are typically employed. Of the poly(N-vinyl lactam) terpolymers, the vinylpyrrolidone, vinylcaprolactam, dimethylaminoethyl methacrylate terpolymers are typically used. A variety of polyvinylpyrrolidones are commercially available.

Hydrogels are stable and maintain their physical integrity after absorbing large quantities of liquid. The gels can be sterilized by radiation sterilization, autoclave or exposed to ethylene oxide. The gels are hydrophilic and capable of absorbing many times of their dry weight in water. Wetting, dispersing agents or surfactants as are known in the art may be added. Glycerin in an amount of 0 to 50 wt. %, preferably from about 5 to 40 wt. % may be added to the gel to increase tack, pliability after drying for the gel. Propylene glycol or polyethylene glycol may also be added. Other additives may be combined with the hydrogels including organic salts, inorganic salts, alcohols, amines, polymer lattices, fillers, surfactants, pigments, dyes, fragrances, etc., among other components described herein.

Of course, any composition having at least secreted products from umbilical cord stem cells can also include one or more other components, in addition to the carriers and excipients mentioned herein. Such at least one additional component can be a topically active agent, that can work additively or synergistically with the secreted products embodied herein. By way of non-limiting example, in addition to sunscreens as described above, agents such as retinoids, steroids, or analgesics or other anti-inflammatories.

In certain embodiments, the formulation embodied here is in the form of a cosmetic, skin care, or other over-the-counter product. In other embodiments, the formulation is an ethical item requiring a prescription by a health care professional. In certain embodiments, the ethical formulation comprises a higher concentration of secreted products than the non-prescription formulation. In other embodiments, various strengths or concentrations of secreted products in a formulation are available, such that the health care professional can match a particular skin condition with a suitable formulation, frequency of application, duration of treatment course, for example, to optimize the desired effect for a particular patient, his or her condition, and compliance with instructions for use.

In one embodiment, treatment of skin comprises treating aging, wrinkling, pigmentation, viscoelasticity, thickness, by way of non-limiting examples.

In another embodiment, a method for treating or preventing undesirable or adverse changes to skin is provided comprising applying to skin a topical composition comprising secreted products from human umbilical cord stem cells, and a dermatologically suitable carrier therefor.

A composition as described herein benefits at least one of the adverse characteristics of skin, such as but not limited to aging, fine wrinkling, furrowing, hyperpigmentation, loss of elasticity, loss of thickness, etc. In other embodiments, the compositions herein are useful for stimulating cellular growth and/or collagen production in skin. Such increased cell growth and/or increased collagen production can help regulate or rejuvenate mammalian skin. The compositions can be used for both prophylactic and therapeutic treatment of skin conditions, such as thickening of skin (i.e., building the epidermis and/or dermis layers of the skin and where applicable the keratinous layers of the nail and hair shaft), preventing and/or retarding atrophy of mammalian skin, preventing and/or retarding the appearance of spider vessels and/or red blotchiness on mammalian skin, preventing and/or retarding the appearance of dark circles under the eye of a mammal, preventing and/or retarding sallow-colored mammalian skin, preventing and/or retarding sagging of mammalian skin, softening and/or smoothing lips, hair and nails of a mammal, preventing and/or relieving itch of mammalian skin, regulating skin texture (e.g. wrinkles and fine lines), and improving skin color (e.g. redness, freckles). As noted above, frequently more than one of the aforementioned conditions affect and individual and the methods and compositions embodied herein are intended to treat one, two, or multiple conditions, and in another embodiment, with the same formulation.

Typically the methods herein are applied to the face, neck, or hands, but may be used on skin anywhere on the body. In other embodiments, the methods embodied herein are useful for non-human mammals, such as domestic and livestock animals.

A topical composition comprising secreted products of umbilical cord stem cells is provided for application to the skin by the user, such as but not limited to application by hand, after extruding the composition from a tube, bottle or other suitable container. In alternate embodiments, the topical composition can be sprayed on from a aerosol can or spray bottle, painted or brushed onto the skin using an applicator, by way of non-limiting examples. A solid or semisolid hydrogel formulation comprising a composition of the invention may be used to cover the portion of skin to which compounds are to be delivered. A topical controlled release delivery system or device can also be used, to deliver the secreted products over time. Application can be performed at a frequency specified on the package, or in accordance with a provider's recommendation or prescription. The amount of secreted products present in the topical formulation will depend on the potency, frequency of application, nature of the carrier, solubility, and other factors readily determinable based on the desired outcome and selection of carrier. Typically, secreted products can be present in a formulation between 0.001% and 10% (w/v). Individual components can each be present from about 0.000001 to 1%.

The following examples are intended to illustrate but not limit the invention. While they are typical of those that might be used, other procedures known to those skilled in the art may alternatively be used.

### EXAMPLE 1

### Secreted Products from Umbilical Cord Stem Cells

Frozen human umbilical cord blood mononuclear cells were received from Cambrex, thawed according to manufacturer's recommendation and placed into 3 T75 flasks with 12 ml each of USSC media (low glucose DMEM with Glutamax, Invitrogen 10567-014), 10⁻⁷ M dexamethasone (Sigma), 100 U/ml penicillin and 0.1 mg/ml streptomycin. Media was changed after 48 hrs then every 2-3 days following. On day 14, proliferating cells were passaged 1:3 into new flasks by using 0.25% trypsin/EDTA and neutralized by trypsin neutralization solution. Every 5 to 7 days cells were similarly passaged. Aliquots of these umbilical cord stem cells (herein abbreviated “UCSC”) were cryopreserved at several passages.

The phenotype of passage 5 UCSC was CD31 (2%), CD34 (0%), CD44 (97%), CD50 (0%), CD71 (47%), CD90, (96%), CD 106 (0%).

A formulation is prepared from conditioned medium from UCSC as described above, and further comprises a hydrogel wound dressing-type polymer. Overnight application of the hydrogel formulation to the skin is found to improve its appearance.

In another example, a formulation for topical application is prepared from umbilical cold stem cells that are CD34^{pos}. Overnight application of the formulation to the skin is found to improve its appearance.

### EXAMPLE 2

### Components from Secreted Products for Skin Care Uses

Among a large number of components identified in secreted products from umbilical cord blood stem cells, the following components were found to provide the ability to induce tumor-like growth of skin cells (without inducing tumorigenesis) and increase the number of keratinocytes in the skin. Such properties are useful for enhancing adverse properties of the skin as discussed hereinabove. The components are elastase 2A; prostaglandin I2; and prostaglandin E2; adam metallopeptidase with thrombospondin type 1 motif 5; bone morphogenetic protein 1; bone morphogenetic protein 6; chemokine (C—C motif) ligand 2; chemokine (C—C motif) ligand 20; chemokine (C—X—C motif) ligand 1; chemokine (C—X—C motif) ligand 2; chemokine (C—X—C motif) ligand 3; chemokine (C—X—C motif) ligand 5; chemokine (C—X—C motif) ligand 6; chemokine (C—X—C motif) ligand 9; colony stimulating factor 2; colony stimulating factor 3; gremlin 1, cysteine knot superfamily, homolog (Xenopus laevis); gremlin 2, cysteine knot superfamily, homolog (Xenopus laevis); heparin-binding EGF-like growth factor; natriuretic peptide precursor B; pleiotrophin; pre-B-cell colony enhancing factor 1; tumor necrosis factor (ligand) superfamily, member 4; and tumor necrosis factor receptor superfamily, member 11b.

Also present were culture medium components, including MgSO₄ (anhydrous); CaCl₂ (anhydrous); KCl; NaCl; NaHCO₃; NaH₂PO₄.H₂O; L-alanine; L-arginine.HCl; L-asparagine.H₂O; L-aspartic acid; L-cysteine.HCl.H₂O; L-cystine.2HCl; L-glutamic acid; L-glutamine; glycine; L-histidine.HCl.H₂O; L-isoleucine; L-leucine; L-lysine.HCl; L-methionine; L-phenylalanine; L-proline; L-serine; L-threonine; L-tryptophan; L-tyrosine.2Na.2H₂O; L-valine; ascorbic acid; biotin; D-calcium pantothenate; i-inositol; nicotinamide; pyridoxine.HCl; riboflavin; thiamine.HCl; vitamin B12; choline chloride; folic acid; D-glucose; lipoic acid; sodium pyruvate; thymidine; adenosine; cytidine; guanosine; uridine; 2′-deoxyadenosine; 2′-deoxycytidine.HCl; 2′-deoxyguanosine; and fetal calf serum.

### EXAMPLE 3

### Components from Secreted Products for Cosmetic Uses

Among a large number of components found in secreted products from umbilical cord blood stem cells, the following were found to provide the ability to increase type V collagen production in the skin, increases vascularization of the skin; or increases glandular secretions in the epidermal layer of the skin. Such properties are useful for treating adverse properties of the skin, and can be provided in the form of a cosmetic product. The components are elastase 2A; prostaglandin I2; prostaglandin E2; amphiregulin; fibroblast growth factor 2; fibroblast growth factor 7; G protein-coupled receptor, family C, group 5, member B; and GABA(a) receptor-associated protein like 1.

The foregoing composition also comprised the components of umbilical cord blood stem cell culture medium from which the individual components were identified. Those culture medium components are described in Example 2.

While certain features have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A composition for application to the skin for the prevention or treatment of a skin condition, the composition comprising secreted products from umbilical cord stem cells, and a topically suitable carrier therefor.

2. The composition of claim 1 wherein the umbilical cord blood stem cells are CD34^{pos} stem cells.

3. The composition of claim 1 wherein the umbilical cord blood stem cells are adherent, CD45^{neg}, HLA class II^{neg} stem cells.

4. The composition of claim 3 wherein said adherent, CD45^{neg}, HLA class II^{neg} stem cells are CD34^{neg}, CD106^{neg}, CD44^{pos} and CD90^{pos}.

5. The composition of claim 1 wherein the skin condition is consequences of aging, fine wrinkling, furrowing, hyperpigmentation, loss of elasticity, loss of thickness, or any combination of any of the foregoing.

6. The composition of claim 1 wherein the carrier comprises a liquid, cream, aerosol, lotion, or hydrogel.

7. The composition of claim 1 wherein the secreted products are present in the composition at a concentration of about 0.001% to about 10%.

8. A method for treating skin comprising applying to skin a topical composition comprising secreted products from human umbilical cord stem cells, and a dermatologically suitable carrier therefor.

9. The method of claim 8 wherein the umbilical cord blood stem cells are CD34^{pos} stem cells.

10. The method of claim 8 wherein the umbilical cord blood stem cells are adherent, CD45^{neg}, HLA class II^{neg} stem cells.

11. The method of claim 10 wherein said adherent, CD45^{neg}, HLA class II^{neg} stem cells are CD34^{neg}, CD106^{neg}, CD44^{pos} and CD90^{pos}.

12. The method of claim 8 wherein the skin condition is consequences of aging, fine wrinkling, furrowing, hyperpigmentation, loss of elasticity, loss of thickness, or any combination of any of the foregoing.

13. The method of claim 8 wherein the carrier comprises a liquid, cream, aerosol, lotion, or hydrogel.

14. The method of claim 8 wherein the secreted products are present in the composition at a concentration of about 0.001% to about 10%.

15. A composition for application to the skin for the prevention or treatment of an adverse skin condition, the composition comprising elastase 2A; prostaglandin I2; prostaglandin E2; adam metallopeptidase with thrombospondin type 1 motif 5; bone morphogenetic protein 1; bone morphogenetic protein 6; chemokine (C—C motif) ligand 2; chemokine (C—C motif) ligand 20; chemokine (C—X—C motif) ligand 1; chemokine (C—X—C motif) ligand 2; chemokine (C—X—C motif) ligand 3; chemokine (C—X—C motif) ligand 5; chemokine (C—X—C motif) ligand 6; chemokine (C—X—C motif) ligand 9; colony stimulating factor 2; colony stimulating factor 3; gremlin 1, cysteine knot superfamily, homolog (Xenopus laevis); gremlin 2, cysteine knot superfamily, homolog (Xenopus laevis); heparin-binding EGF-like growth factor; natriuretic peptide precursor B; pleiotrophin; pre-B-cell colony enhancing factor 1; tumor necrosis factor (ligand) superfamily, member 4; and tumor necrosis factor receptor superfamily, member 11b.

16. The composition of claim 15 further comprising MgSO₄ (anhydrous); CaCl₂ (anhydrous); KCl; NaCl; NaHCO₃; NaH₂PO₄.H₂O; L-alanine; L-arginine.HCl; L-asparagine.H₂O; L-aspartic acid; L-cysteine.HCl.H₂O; L-cystine.2HCl; L-glutamic acid; L-glutamine; glycine; L-histidine.HCl.H₂O; L-isoleucine; L-leucine; L-lysine.HCl; L-methionine; L-phenylalanine; L-proline; L-serine; L-threonine; L-tryptophan; L-tyrosine.2Na.2H₂O; L-valine; ascorbic acid; biotin; D-calcium pantothenate; i-inositol; nicotinamide; pyridoxine.HCl; riboflavin; thiamine.HCl; vitamin B12; choline chloride; folic acid; D-glucose; lipoic acid; sodium pyruvate; thymidine; adenosine; cytidine; guanosine; uridine; 2′-deoxyadenosine; 2′-deoxycytidine.HCl; 2′-deoxyguanosine; and fetal calf serum.

17. A method for prevention or treatment of an adverse skin condition comprising applying to the skin a topical formulation comprising a composition of claims 15 or 16.

18. The method of claim 17 wherein the adverse skin condition is consequences of aging, fine wrinkling, furrowing, hyperpigmentation, loss of elasticity, loss of thickness, or any combination of any of the foregoing.

19. The method of claim 17 wherein the composition induces tumor-like growth of skin cells (without inducing tumorigenesis) or increases the number of keratinocytes in the skin.

20. A method for inducing tumor-like growth of skin cells (without inducing tumorigenesis) or increasing the number of keratinocytes in the skin comprising applying to the skin a topical formulation of any one of claims 1-7 or 15-16.

21. A composition for application to the skin for the prevention or treatment of an adverse skin condition, the composition comprising elastase 2A; prostaglandin I2; prostaglandin E2; amphiregulin; fibroblast growth factor 2; fibroblast growth factor 7; G protein-coupled receptor, family C, group 5, member B; and GABA(a) receptor-associated protein like 1.

22. The composition of claim 21 further comprising MgSO₄ (anhydrous); CaCl₂ (anhydrous); KCl; NaCl; NaHCO₃; NaH₂PO₄.H₂O; L-alanine; L-arginine.HCl; L-asparagine.H₂O; L-aspartic acid; L-cysteine.HCl₂O; L-cystine.2HCl; L-glutamic acid; L-glutamine; glycine; L-histidine.HCl.H₂O; L-isoleucine; L-leucine; L-lysine.HCl; L-methionine; L-phenylalanine; L-proline; L-serine; L-threonine; L-tryptophan; L-tyrosine.2Na.2H₂O; L-valine; ascorbic acid; biotin; D-calcium pantothenate; i-inositol; nicotinamide; pyridoxine.HCl; riboflavin; thiamine.HCl; vitamin B12; choline chloride; folic acid; D-glucose; lipoic acid; sodium pyruvate; thymidine; adenosine; cytidine; guanosine; uridine; 2′-deoxyadenosine; 2′-deoxycytidine.HCl; 2′-deoxyguanosine; and fetal calf serum.

23. A method for prevention or treatment of an adverse skin condition comprising applying to the skin a topical formulation comprising a composition of any one or claims 21-22.

24. The method of claim 23 wherein the adverse skin condition is consequences of aging, fine wrinkling, furrowing, hyperpigmentation, loss of elasticity, loss of thickness, or any combination of any of the foregoing.

25. The method of claim 23 wherein the composition increases type V collagen production in the skin, increases vascularization of the skin; or increases glandular secretions in the epidermal layer of the skin.

26. A method for increasing type V collagen production in the skin, increasing vascularization of the skin; or increasing glandular secretions in the epidermal layer of the skin comprising applying to the skin a topical formulation of any one of claims 1-7 or 21-22.
